(19) United States
(12) Patent Application Publication
(10) Pub. No.: US 2007/0224269 A1
(43) Pub. Date: Sep. 27, 2007

(54) Controlled Release Pharmaceutical Formulation

(76) Inventors: Orapin P. Rubino, Towaco, NJ (US); David M. Jones, Ramsey, NJ (US); Oliver Mueller, Kinnelon, NJ (US); Robert Femia, Kinnelon, NJ (US); Narayan Ragunathan, West Nyack, NJ (US); Iosif Oscar Fishkis, White Palins, NY (US)

Correspondence Address: HEDMAN & COSTIGAN P.C., 1185 AVENUE OF THE AMERICAS, NEW YORK, NY, 10036, US

(21) Appl. No.: 11/628,891

(22) PCT Filed: Jun. 8, 2005
(86) PCT No.: PCT/US05/20106

§ 371 Date: Dec. 7, 2006

Related U.S. Application Data
(60) Provisional application No. 60/578,934, filed on Jun. 10, 2004.

(51) Int. Cl. A61K 9/32

(52) U.S. Cl. 424/465

(57) ABSTRACT

The invention proves a novel compressed tablet of a pharmaceutical compound and a method of making a tablet of a pharmaceutical compound which are based on uncoated pellets containing a pharmaceutical compound that are dispersed in a matrix which comprises said pellets and a swellable polymer which is compressed into a tablet that is coated with an enteric polymer.

### BACKGROUND OF THE INVENTION

Oral solid dosage forms have been described in the prior art which are based on pellets which are dispersed in a matrix which is compressed into a tablet. U.S. Pat. No. 5,637,320 describes a formulation where pellets of naproxen are coated with a multilayer membrane which controls the release of the naproxen. The present applicant has discovered that it is not necessary to provide coated pellets in a compressed tablet matrix to obtain controlled release properties of a drug contained in the pellets if the matrix is formulated to contain a swellable pharmaceutical polymer.

Other oral solid dosage forms of oxybutynin chloride are commercially available such as Ditropan XL which comprises an osmotically active bilayer core surrounded by a semi-permeable membrane. A laser drilled hole is provided in the osmotic dosage form on the drug layer side for allowing the drug to be pushed out of the dosage form through the laser drilled hole.

The applicants have discovered that if uncoated drug pellets containing a pharmaceutical compound are dispersed in a controlled release polymer containing matrix which is compressed into a tablet which is subsequently enteric coated, the resulting dosage form can be prepared in such a way that it is usable as a once a day dosage form. In addition, an oxybutynin chloride matrix tablet which contains uncoated pellets according to the invention may also be formulated in such a manner that the dosage form is bioequivalent to the commercially available osmotic dosage form. commercially available osmotic dosage form.

Typically, in the prior art, pellets have been used in formulations for sustained or controlled release where the pellets are coated with controlled or modified release polymers to obtain a sustained or controlled release dosage form. It has been discovered that uncoated drug pellets, when combined with a matrix comprising a swellable or controlled release polymer will provide extended release of the drug oxybutynin chloride.

A compressed tablet, made with a controlled release polymer in the matrix, which is preferably a carbomer, in combination with an uncoated pharmaceutical pellets can provide a zero-order release formulation containing a pharmaceutical compound suitable for once a day administration if the tabletted formulation is coated with an enteric coating. The enteric coated extended release tablets, according to the invention, when tested in dissolution media that represents conditions in the stomach (two hours in acid media) followed by a media change to a media that represents conditions in the gastrointestinal tract (pH 6.8 phosphate buffer), will provide a zero-order release of the pharmaceutical compound over a period of time which permits once a day dosing.

### SUMMARY OF THE INVENTION

The invention provides a novel compressed tablet formulation of a pharmaceutical compound which comprises uncoated pellets containing said pharmaceutical compound which are dispersed in a matrix which comprises said pellets and a swellable polymer which is compressed into a tablet that is coated with an enteric polymer.

Accordingly it is an object of the invention to provide a zero order controlled release formulation of a pharmaceutical compound.

It is also an object of the invention to provide a zero order controlled release formulation of a pharmaceutical compound which will permit once a day dosing.

These and other objects of the invention will be apparent from the specification.

As used herein the term “pellet” means a substantially spherically shaped particle having a aspect ratio (a ratio of the length of the pellet divided by the width found at an angle of 90° in respect to the length) which is less than about 1.4, more preferably less than about 1.3, even more preferably less than about 1.2, especially preferably less than about 1.1, and most preferably less than about 1.05.

### BRIEF DESCRIPTION OF THE DRAWINGS

FIG. 1 is a dissolution profile of the oxybutynin tablets of Example 1 which are not enteric coated (Dissolution Media:pH6.8 Phosphate Buffer).

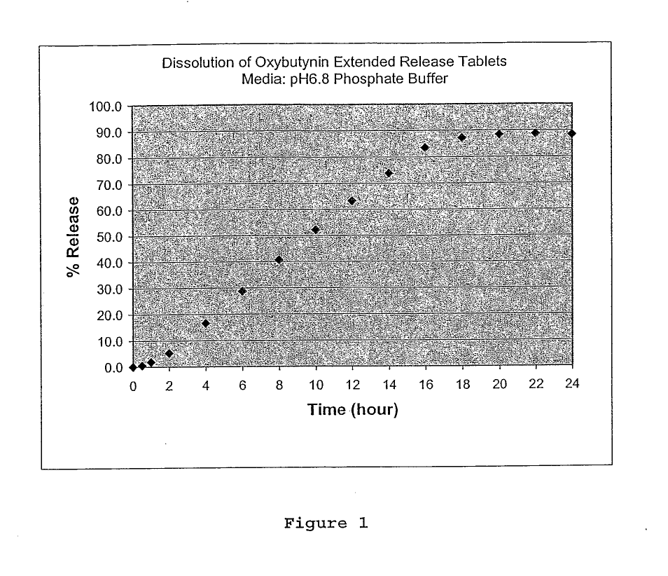

FIG. 2 is a dissolution profile of the oxybutynin tablets of Example 1 which are enteric coated (Dissolution Media: 0.1N HCl 2 hours/pH6.8 Phosphate Buffer 22 hours).

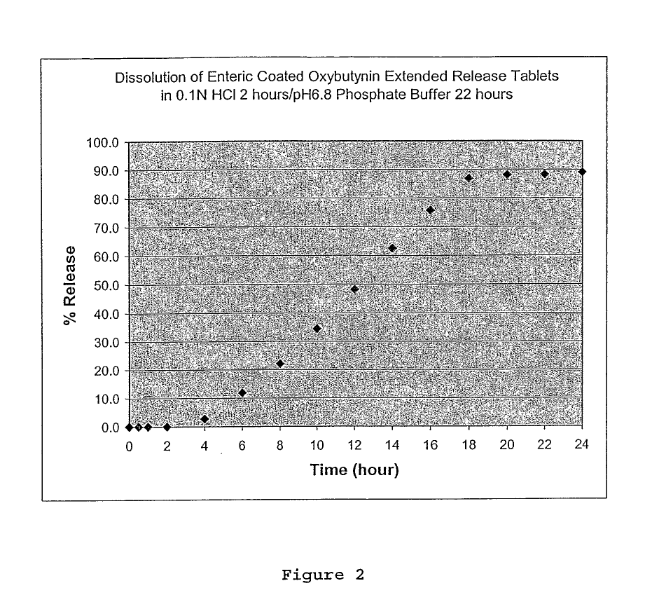

FIG. 3 is a dissolution profile of the oxybutynin tablets of Example 2 which are subcoated and enteric coated (Dissolution Media: 0.1N HCl 2 hours/pH6.8 Phosphate Buffer 22 hours).

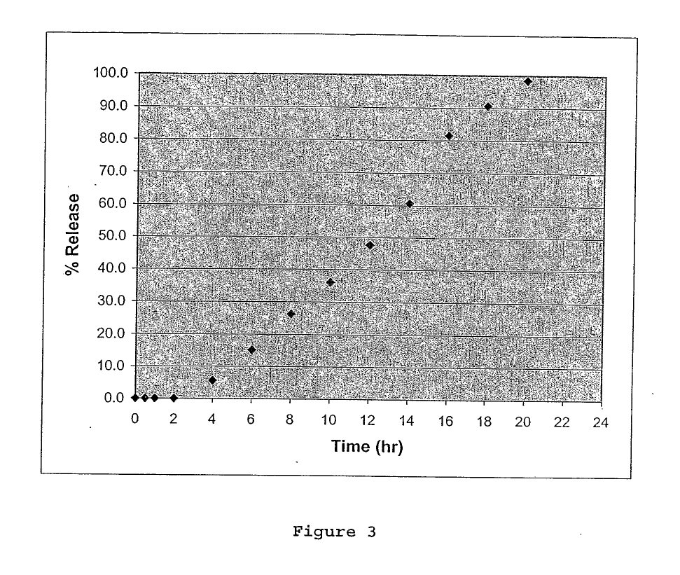

### DETAILED DESCRIPTION OF THE INVENTION

The term “uncoated pellet” is used to define pellets that have no coating or a coating that has no effect on the release rate of a pharmaceutical compound that is contained in the pellet. Thus in an preferred embodiment, the pellets will have no coating but it is possible to utilize pellets that have highly water soluble or highly permeable coatings that behave as if they are water soluble by not affecting the release rate of drug from a pellet. Generally, the pellets of a pharmaceutical compound will release not less than 70 wt % of pharmaceutical compound when tested in 900 ml of deionized water at 37° C., at 50-100 rpm in a USP Type 1 apparatus (basket) in two hours.

The uncoated pellets of the invention may be made using any conventional pelletizing process. It is contemplated that conventional layering of drugs on inert cores such as sugar spheres (i.e. sucrose-starch non-pareils), microcrystalline cellulose spheres (i.e. Cellets), or other solid cores such as glass beads and the like using a liquid solution/suspension system or a powder layering system which places active drugs on to an inert core; and extrusion spheronization of pellets containing a binder and/or an active drug. In addition the procedures of U.S. Pat. No. 6,354,728 may be used to make pellets suitable for use in the invention.

Procedures for the making of pellets by extrusion-spheronization are well known in the art. A pharmaceutically active compound and any inactive ingredients (excipients, binders etc.) are pre-mixed, then wetted with water, in a high shear mixer. The damp mass is then transferred into an extruder where it is forced through a screen or die plate, where it forms an essentially solid, cylindrical extrudate of uniform shape and size. The size of the opening in the screen or die dictate resultant pellet size. The extrudate is fed onto a rotating disk, which may be smooth or may contain a grid (waffled, grooved etc.). The extrudate breaks into small cylinders, which in time are rounded into spherically shaped solids. Subsequently, the pellets are dried to the desired residual moisture content, typically in a fluid bed dryer. Any oversized or undersized product is removed by sieving, and the resulting pellets have a narrow size distribution.

The technique of layering an active drug onto to solid core by layering is well known in the art. In solution or suspension layering, a pharmaceutically active compound and any inactive ingredients (excipients, binder etc.) are suspended or dissolved in water or an organic solvent. The resulting liquid is sprayed onto the outside of a core particle, which may be a non-pareil sugar seed (sugar sphere), microcrystalline cellulose pellets (such as Cellets or Celphere) and the like, to the desired potency. Solution or suspension layering may be conducted using a wide variety of process techniques, but a preferred method is by fluidized bed and more preferably the Wurster bottom spray method. When the desired potency has been achieved, pellets are dried to the desired residual moisture content. Any oversized or undersized product is removed by sieving, and the resulting pellets are narrow in size distribution.

Powder layering involves the application of a dry powder to some type of core material. The powder may consist entirely of a pharmaceutical compound, or may include excipients such as a binder, flow aid, inert filler, and the like. Powder layering may be conducted using a wide variety of processing techniques, but a preferred method is by rotary fluidized bed. A pharmaceutically acceptable liquid, which may be water, organic solvent, with or without a binder and/or excipients, is applied to some type of core material while applying the dry powder until the desired potency is achieved. When the desired potency has been achieved, the pellets may be seal coated to improve their strength, and are then dried to the desired moisture content. Any oversized or undersized product is removed by sieving, and the resulting pellets are narrow in size distribution.

An apparatus suitable for making pellets is disclosed in U.S. Pat. No. 6,354,728, which is incorporated by reference. This device comprises a rotor located in a chamber such that an annular gap exists between the rotor and the inner wall of said chamber. Alternatively or in addition, the rotor may contain openings in its surface allowing a gas to pass through.

The gas stream, through the openings in the rotor, may be directed such that forces acting on the pellets being formed are reduced or increased. For instance, a gas may be led through openings in the rotor from below to reduce interactions between pellets and the rotor surface as well as among the pellets. This will reduce the densification of adhering powder particles. The quantity and flow rate of the gas which is passed through the bed of the pellets should not result in a significant fluidization of the pellet bed.

The degree of densification of the powdered pharmaceutical compound will also be influenced by the composition of the pellets being formed. One aspect of the composition of the pellets being formed is their liquid content. A higher liquid content will generally lead to a higher plasticity allowing a more effective densification. However, it has to be noted that, by the process of the invention, the degree of densification can be varied for a given composition by regulating the energy uptake of the pellets being formed when these pellets are subjected to a rolling movement, as described above.

The degree of densification of the powdered pharmaceutical compound and any excipients/binder in the pellets made for use in the invention may be determined by the absolute porosity of the formed pellet or layer. A high porosity corresponds to a low degree of densification, and vice versa.

The porosity may be visualized by microscopic techniques, for instance by scanning electron microscopy. Alternatively, the porosity may be determined by mercury intrusion.

The degree of densification will also be reflected in the density of the pellets prepared. A higher degree of densification leads to a higher density. The achieved absolute porosity, i.e. the percentage of the total void space with respect to the bulk volume, may vary between 0.5 and 30%. Preferably, the absolute porosity has a value of from 1 to 20%, more preferably of from 1 to 10%, and especially from 2 to 10%.

The pellets of the pharmaceutical compound may be made in such a manner that the degree of densification is such that a gradient of the degree of densification in a radial direction is achieved or separate concentric zones having varying levels of densification may be formed on each pellet, either in the core or in one or more layers. The degree of densification may be controlled so that at least one layer has a density that is lower than the bulk density of the starting powder.

Generally the pellets of the pharmaceutical compound according to the invention will have a diameter of from 0.01 to 2 mm, such as from 0.1 to 1.25 mm. The layer or layers will each have a layer thickness of from 0.005 to 0.01 mm, such as from 0.05 to 0.75 mm. The pellets prepared according to the invention have a narrow particle size distribution such that a maximum of 20% by weight of the pellets have a diameter deviating from the average diameter of all by more than 20%. Preferably, a maximum of 10% by weight of the pellets have a diameter deviating from the average diameter of all, by more than 20%. Further preferably, a maximum of 20% by weight of the pellets have a diameter deviating from the average diameter of all pellets by more than 10% by weight. An especially preferred pellet product has a particle size distribution such that a maximum of 10% by weight of the pellets have a diameter deviating from the average diameter of all pellets by more than 10% by weight. All percents by weight are based on the total weight of the pellets.

A preferred method of preparing pellets of a pharmaceutical compound comprises:

(a) forming a powder mixture which comprises a binder such as microcrystalline cellulose and oxybutynin chloride;

(b) feeding said powder mixture which is optionally pre-wetted with from 0-60 wt % of a pharmaceutically acceptable liquid diluent, based on the total weight of the powder mixture and the pharmaceutically acceptable diluent, to an operating apparatus which comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing said powdered excipient, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said powdered product which is circulated by kinetic energy by said rotor under the influence of kinetic energy, moves from said rotor to an inside surface of said guide vanes before falling back onto said rotor;

(c) rotating said rotor, while feeding air and spraying a pharmaceutically acceptable liquid into said rotor chamber for a sufficient amount of time to form solid pellets having a desired diameter; and

(d) feeding a sufficient amount of a substantially dry, free flowing inert powder which forms a non-tacky surface when placed in contact with water to provide on said pellets an outer zone comprising a layer formed from said substantially dry, free flowing inert powder.

The pellets of a pharmaceutical compound when made in apparatus of U.S. Pat. No. 6,354,728, which describes the use of a rotating device that propels the powder particles onto a tangentially arranged surface which causes the powder particles to roll on said tangentially arranged surface. This process results in pellets having a controlled density, for instance highly dense pellets. The pellets may be: adapted to contain high levels of a pharmaceutical compound, i.e. 1-95 wt %, and preferably from 5-90 wt % based on the total weight of the pellet with the balance being a suitable pharmaceutical excipient and/or binder. The pellets may be manufactured with a narrow size distribution without the need to carry out any substantial separation step.

The pellets for use in the invention may be prepared using an apparatus which propels particles against a tangentially arranged inner wall in such a manner that a rolling motion is imparted to the moving pellets. A liquid is fed into an apparatus such as the apparatus disclosed in U.S. Pat. No. 6,449,869 which is adapted to allow for the introduction of a pharmaceutical compound in powder form during the operation of the apparatus. In one embodiment of the invention, the process of the invention involves the introduction of an oxybutynin chloride containing powder as a final step in the process in order to control and/or terminate pellet growth as well as assisting in the drying, rounding and smoothing of the pellets. The preferred apparatus is described in U.S. Pat. Nos. 6,449,869 and 6,354,728, both of which are incorporated by reference.

When core layered pellets are used, such as sugar spheres, from 20% to 99 wt %, preferably 30-80 wt % of the pharmaceutical compound, may be layered onto the sugar sphere based on the total weight of the sugar sphere and the pharmaceutical compound. If desired a pharmaceutical excipient and/or binder may be used in the layering process in an amount which will be from 1-20 wt %, preferably 1-10 wt % of the total weight of the pharmaceutical compound and the excipient.

The pharmaceutically acceptable liquid which is used in the formation of the pellets may comprise one or more components selected from the group consisting of a pharmaceutical compound, binders, diluents, disintegrants, lubricants, flavoring agents, coloring agents, surfactants, anti-sticking agents, osmotic agents, matrix forming polymers, film forming polymers, release controlling agents, stabilizers and mixtures thereof, in dissolved, suspended or dispersed form. Generally, only selected components will be employed to achieve the desired result for a given formulation. The particular formulation will determine if, when and how the listed components are added.

The active pharmaceutical compounds that can be delivered includes inorganic and organic compounds without limitation, including drugs that act on the peripheral nerves, adrenergic receptors, cholinergic receptors, nervous system, skeletal muscles, cardiovascular system, smooth muscles, blood circulatory system, synaptic sites, neuroeffector junctional sites, endocrine system, hormone systems, immunological system, reproductive system, skeletal system, autacoid systems, alimentary and excretory systems, inhibitory of autocoid systems, alimentary and excretory systems, inhibitory of autocoids and histamine systems. The active drug that can be delivered for acting on these recipients include anticonvulsants, analgesics, anti-inflammatories, calcium antagonists, anesthetics, antimicrobials, antimalarials, antiparasitic, antihypertensives, antihistamines, antipyretics, alpha-adrenergic agonist, alpha-blockers, biocides, bactericides, bronchial dilators, beta-adrenergic blocking drugs, contraceptives, cardiovascular drugs, calcium channel inhibitors, depressants, diagnostics, diuretics, electrolytes, hypnotics, hormonals, hyperglycemics, muscle contractants, muscle relaxants, ophthalmics, psychic energizers, parasympathomimetics, sedatives, sympathomimetics, tranquilizers, urinary tract drugs, vaginal drugs, vitamins, nonsteroidal anti-inflammatory drugs, angiotensin converting enzymes, polypeptide drugs, and the like.

Exemplary drugs that are very soluble in water and can be delivered by the pellets of this invention include prochlorperazine, ferrous sulfate, aminocaproic acid, potassium chloride, mecamylamine hydrochloride, procainamide hydrochloride, amphetamine sulfate, amphetamine hydrochloride, isoproteronol sulfate, methamphetamine hydrochloride, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, cimetidine hydrochloride, theophylline cholinate, cephalexin hydrochloride, oxybutynin chloride and the like.

Exemplary drugs that are poorly soluble in water and that can be delivered by the particles of this invention include diphenidol, meclizine hydrochloride, omeprazole, esomeprazole, lansoprazole, pantoprazol, prochlorperazine maleate, phenoxybenzamine, thiethylperzine maleate, anisindone, diphenadione, erythrityl tetranitrate, digoxin, isoflurophate, acetazolamide, methazolamide, bendro-flumethiazide, chlorpropamide, tolazamide, chlormadinone acetate, phenaglycodol, allopurinol, aluminum aspirin, methotrexate, acetyl sulfisoxazole, erythromycin, progestins, progestational, corticosteroids, hydrocortisone hydrocorticosterone acetate, cortisone acetate, triamcinolone, methyltestosterone, 17 beta-estradiol, ethinyl estradiol, ethinyl estradiol 3-methyl ether, prednisolone, 17 betahydroxyprogesterone acetate, 19 non-progesterone, norgesterel, norethindrone, norethisterone, norethiederone, progesterone, norgesterone, norethynodrel, and the like.

Examples of other drugs that can be formulated according to the present invention include aspirin, indomethacin, naproxen, fenoprofen, sulindac, indoprofen, nitroglycerin, isosorbide dinitrate, timolol, atenolol, alprenolol, cimetidine, clonidine, imipramine, levodopa, chloropromazine, methyldopa, dihydroxyphenylalamine, pivaloyloxyethyl ester of alpha-methyldopa hydrochloride, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, captopril, madol, propranolol hydrochloride, quanbenz, hydrochlorothiazide, ranitidine, flurbiprofen, fenbufen, fluprofen, tolmetin, alolofenac, mefanamic, flufenamic, difuninal, nimodipine, nitrendipine, nisoldipine, nicardipine, felodipine, lidoflazine, tiapamil, gallopamil, amlodipine, mioflazine, lisinolpril, enalapril, captopril, ramipril, endlapriate, famotidine, nizatidine, sucralfate, etintidine, tertatolol, minoxidil, chlordiazepoxide, chlordiazepoxide hydrochloride, diazepam, amitriptylin hydrochloride, impramine hydrochloride, imipramine pamoate, enitabas, buproprion, and the like.

Other examples of pharmaceutical compounds include water soluble vitamins such as the B Vitamins, Vitamin C and the oil soluble vitamins such as Vitamin A, D, E and K. Neutraceuticals such as chondroitin, glucosamine, St. John's wort, saw palmetto and the like may also be formed into pellets according to the present invention

Suitable binders include materials that impart cohesive properties to the pharmaceutical compound when admixed dry or in the presence of a suitable solvent or liquid diluent. These materials commonly include starches such as pregelatinized starch, gelatin, and sugars such as sucrose, glucose, dextrose, molasses and lactose. Natural and synthetic gums include acacia, sodium alginate, extract of Irish moss, panwar gum, ghatti gum, mucilage of isapol husks, carboxymethyl cellulose, methylcellulose, microcrystalline cellulose, polyvinylpyrrolidone e.g. povidone U.S.P K30, Veegum, and larch arabogalactan. Binders are used in an effective amount, e.g. 1 to 10 wt %, based on the total weight of liquid and binder to cause a sufficient degree of agglomeration of the oxybutynin chloride in order to allow the rapidly formation of stable particles.

Examples of pharmaceutical excipients or diluents for use in making pellets of a pharmaceutical compound include water soluble and water insoluble materials. Examples of useful materials include microcrystalline cellulose, dicalcium phosphate, calcium sulfate, talc, an alkali metal stearate, silicon dioxide and calcium carbonate.

As noted above, pellets suitable for use in the invention may be made by using an apparatus that is described in U.S. Pat. No. 6,354,728. That apparatus comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing said powdered excipient, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said powdered product which is circulated by kinetic energy by said rotor under the influence of kinetic energy, moves from said rotor to an inside surface of said guide vanes before it falls back onto said rotor.

When the desired pellet size is substantially achieved, it is preferred to feed dry powder to the apparatus and the apparatus is allowed to run for a period of 3 to 15 minutes, and preferably 5 to 10 minutes to complete the formation of the pellets.

It is also contemplated that some additional drying at a temperature of from about 30 to 100° C., and preferably from about 40 to 90° C. until the moisture content is from 1 to 10 wt %, based on the total weight of the pellets.

The matrix forming material may be any swellable matrix forming material that provides in vitro dissolution rates of a biologically active agent within the narrow ranges required to provide the desired plasma level of the oxybutynin chloride over a desired interval which is typically 8 to 24 hours. Most matrix forming materials will also provide for the release of oxybutynin chloride in a pH independent manner. Preferably the matrix is based on a pharmaceutically acceptable, water swellable polymer which forms a controlled release matrix. Suitable water-swellable materials for inclusion in a controlled release matrix are hydrophilic polymers, such as carbomers having a viscosity of 3,000 to 60,000 mPa s as a 0.5%-1% w/v aqueous solution, cellulose ethers such as hydroxypropylcellulose having a viscosity of about 1000-7000 mPa s as a 1% w/w aqueous solution (25° C.), hydroxypropyl methylcellulose having a viscosity of about 1000 or higher, preferably 2,500 or higher to a maximum of 25,000 mPa s as a 2% w/v aqueous solution; polyvinylpyrrolidone having a viscosity of about 300-700 mPa s as a 10% w/v aqueous solution at 20° C. Specifications for these materials are found in the Handbook of Pharmaceutical Excipients, 4th Ed, Rowe et al., Pharmaceutical Press (2003) which is incorporated by reference. Of these polymers, the carbomer polymers are preferred. In particular carbomer polymers are commercially available as Carbopol in powder (Carbopol 971P) or granular form (Carbopol 71G). A blend of carbomer in powder form (e.g. about 0.2 μm average diameter) and carbomer in granular form (e.g. about 180-425 μm average diameter) provides a desirable formulation when blended in a 10-90 wt % to 90-10 wt % ratio(granular/powder) or more preferably in a 30-70 wt % to 70-30 wt % ratio (granular/powder) based on the total weight of the carbomers.

Tablet lubricants include such well known materials as magnesium stearate, stearic acid, calcium stearate, sodium stearyl fumarate, glyceryl palmitostearate, glyceryl behenate, glyceryl monostearate, poloxamer, polyethylene glycol having a weight average molecular weight of 1000-6000 and the like.

The enteric coating polymer may be selected from the group consisting of shellac, methacrylic acid copolymers, (Eudragit S or L) cellulose acetate phthalate, hydroxypropyl methylcellulose phthalate, hydroxypropyl methylcellulose acetate succinate, cellulose acetate trimellitate and polyvinyl acetate phthalate. Poly(methacrylic acid, ethyl acrylate) carboxyl/ester ratio 1:2 wt average mol. weight of about 135,000 which is available as a 30% aqueous dispersion and dissolves at a pH of about 5.5 is preferred (Eudragit L30D-55). The thickness of the coating is selected to provide the desired release rate.

Other auxiliary coating aids such as a minor amount (1-30 wt %, preferably 5-15 wt % based on the total weight of the final coating) of a plasticizer such as acetyltributyl citrate, triacetin, acetylated monoglyceride, rape oil, olive oil, sesame oil, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, polyethyleneglycol (molecular weight of from 380 to 420), propylene glycol and mixtures thereof in combination with an antisticking agent which may be a silicate such as talc. An antisticking agent, such as talc, glyceryl monostearate, magnesium stearate and the like may be added in an amount which is effective to prevent sticking of the pellets. These components may be added to the methacrylic acid copolymer in combination with appropriate solvents.

It may be desirable to increase the stability of the dosage form of the invention by placing a water soluble subcoating on the tablets before they are enteric coated. This separates the matrix materials from the enteric coating and increases the stability of the tablets. Suitable water soluble coatings include low viscosity hydroxy propyl methylcellulose (viscosity of 2.4-60 mPa s as a 2% w/v aqueous solution); low viscosity hydroxypropyl cellulose (viscosity of 75-600 mPa s in a 5-10% aqueous solution at 25° C.) (e.g. Klucel EF and LF) or povidone having a dynamic viscosity of 1-10 mPa s as a 10% aqueous solution at 20° C. Other materials include commercial aqueous formulations of 2-10 wt % low molecular weight hydroxypropyl methylcellulose (Methocel E-5), available as Opadry coatings.

The uncoated pharmaceutical compound pellets are formulated into tablets with the matrix forming polymer using conventional tabletting techniques to provide therapeutic doses which are well known to those who are skilled in the art.

The tablets of the invention may comprise:

| | general | preferred |
|---|---|---|
| pharmaceutical compound | 10-70 wt % | 20-50 wt % |
| swellable polymer | 5-50 wt % | 5-40 wt % |
| pharmaceutical excipient | 25-85 wt % | 30-70 wt % |
| tablet lubricant | 1-10 wt % | 2-5 wt % |
| enteric coating | 2-15 wt % | 3-10 wt % |

(based on the total weight of the tablet)

### DESCRIPTION OF THE PREFERRED EMBODIMENTS

### EXAMPLE 1

A granular form of carbomer (Carbopol 71G) is used to prepare oxybutynin chloride extended release tablets as follows:

| Procedure: | Blend |
|---|---|
| Oxybutynin HCl | 15.0% |
| Microcrystalline cellulose | 56.7% |
| Dicalcium Phosphate | 28.3% |

Load the above ingredients (total weight of blend 16 Kg) in a vertical high shear granulator for 2 min.

Weigh 3.2 Kg of the blend for powder feeding portion Spray 4.5 Kg of water at 500 g/min spray rate, atomization air pressure 2.0 bar.
Discharge the blend from high shear granulator, load the blend into an apparatus as described in U.S. Pat. No. 6,354,728.
Start the apparatus and spray water at 250 g/min.
Process conditions follow:
   Inlet air temperature 17° C.
   Rotor speed 500 rpm initial, reduced to 250 rpm after 1.6 Kg of water applied.
   After 7.1 Kg of water applied, start powder feed at 235 g/min.
   Stop process after 8.6 Kg water is applied.
Discharge the wet pellets. Dry in a fluid bed dryer.

Final moisture 1.71%.

The pellets were sieved to obtain a fraction of 25/35 US Standard mesh or 500-710 microns.

| Tablet formulation: | Quantity |
|---|---|
| Oxybutynin Pellets (15%) | 455.0 g |
| Microcrystalline cellulose | 385.0 g |
| Carbopol 71G | 120.0 g |
| Stearic acid | 40.0 g |
| Total | 1000.0 g |

Carbopol 71G is a granular carbomer having a viscosity of 4000-11000 as a 0.5 w/v % solution in water and a particle size of 180-425μm.

The tablet ingredients are mixed in a 8 qt. V blender and compressed using a 6 station tablet press (Korsch, model PH 106) to make standard concave round 9/32″ tablets.

The dissolution profiles of these tablets, (uncoated oxybutynin chloride pellets made with a granular carbomer matrix) was determined in a USP Type 2 apparatus, using pH 6.8 phosphate buffer at 37° C. and 50 rpm

| Time | % |
|---|---|
| (hour) | Release |
| 0.0 | 0.0 |
| 0.5 | 0.5 |
| 1.0 | 1.8 |
| 2.0 | 5.2 |
| 4.0 | 16.6 |
| 6.0 | 28.7 |
| 8.0 | 40.6 |
| 10.0 | 52.1 |
| 12.0 | 63.2 |
| 14.0 | 73.8 |
| 16.0 | 83.5 |
| 18.0 | 87.1 |
| 20.0 | 88.4 |
| 22.0 | 88.7 |
| 24.0 | 88.4 |

Enteric coating of Oxybutynin Chloride Tablets

When oxybutynin chloride tablets prepared as described above(uncoated oxybutynin chloride pellets in carbomer matrix) was exposed to 0.1N HCl, which simulates conditions in the stomach, the oxybutynin chloride tablets exhibit a very fast rate of release. For example, the tablets tested above in 0.1N HCl released 59 wt % of oxybutynin chloride in 2 hours as compared to a release of 3.5 wt % of oxybutynin chloride in pH 6.8 buffer 2 hours. An enteric coating of the oxybutynin chloride was used to modify the release of oxybutynin in the stomach.

Oxybutynin chloride extended release tablets using formulation specified above (uncoated oxybutynin chloride pellets in 12% granular carbomer (Carbopol 71G), were coated in a perforated pan using the following enteric coating formulation:

| | |
|---|---|
| Eudragit L30D dispersion | 1500.0 g |
| Triethyl citrate | 67.5 g |
| Purified water USP | 728.5 g |
| Total | 2296.0 g |

Coating is performed in a Glatt GC300 coating pan.

Starting tablets: 1.9 Kg of oxybutynin chloride extended release tablets (with 12% granular carbomer).

Samples were taken at various % weight gain and submitted for dissolution testing.

Dissolution Testing of Enteric Coated Oxybutynin Chloride Extended Release Tablets at 15 wt % enteric coating based on total tablet weight using Eudragit L30D (0.1 N HCl for 2 hours, followed by pH 6.8 buffer, using USP apparatus type 2 at 50 rpm)

| Time | % |
|---|---|
| (hour) | Release |
| 0.0 | 0.0 |
| 0.5 | 0.0 |
| 1.0 | 0.0 |
| 2.0 | 0.0 |
| 4.0 | 2.7 |
| 6.0 | 11.8 |
| 8.0 | 22.1 |
| 10.0 | 34.4 |
| 12.0 | 48.2 |
| 14.0 | 62.5 |
| 16.0 | 75.7 |
| 18.0 | 86.8 |
| 20.0 | 88.1 |
| 22.0 | 88.3 |
| 24.0 | 89.1 |

The enteric coated oxybutynin chloride extended release tablets also have zero-order release characteristics with complete release of oxybutynin chloride in 24 hours.

### EXAMPLE 2

A blend of carbomer (Carbopol 971P and Carbopol 71G) is used to prepare oxybutynin chloride extended release tablets as follows:

Pellet Composition:

| | |
|---|---|
| Oxybutynin HCl | 10.0% |
| Microcrystalline cellulose | 60.0% |
| Dicalcium Phosphate | 30.0% |

The pellets were prepared using the procedure described in Example 1.

The pellets were sieved to obtain a fraction of 40/80 US Standard mesh or 180-425 microns.

| Tablet formulation: | Quantity |
|---|---|
| Oxybutynin Pellets | 6.825 Kg |
| Microcrystalline cellulose | 4.275 Kg |
| Carbopol 971P | 0.900 Kg |
| Carbopol 71G | 2.400 Kg |
| Stearic acid | 0.600 Kg |
| Total | 15.000 Kg |

Carbopol 971P is a powdered carbomer having a viscosity of 4000-11000 mPa as a 0.5 w/v % solution in water and a primary particle size of about 0.2 μm in diameter. Carbopol 71G is a granular carbomer having a viscosity of 4000-11000 as a 0.5 w/v % solution in water and a particle size of 180-425 μm.

The tablet ingredients are mixed in a 2 Cu. Ft. V-blender and compressed using a 12 station tablet press (Kikusui, Virgo) to make standard concave round 9/32″ tablets.

The tablets were coated in a 24 inch Perforated coating pan (Compulab) with a subcoat solution (to 2% solid weight gain) and enteric coating solution (to 3% solid weight gain).

Subcoating solution:

| | |
|---|---|
| Opadry Clear | 0.300 Kg |
| Purified water | 2.700 Kg |

Opadry clear is an aqueous solution of low molecular weight HPMC polymer as supplied commercially by Colorcon.

Enteric coating solution:

| | |
|---|---|
| Eudragit L30D-55 | 2.000 Kg |
| Triethyl citrate | 0.090 Kg |
| Talc | 0.060 Kg |
| Purified water | 0.970 Kg |

The dissolution profiles of these tablets, (uncoated oxybutynin chloride pellets made with a blend of Carbopol 971P and 71G, with a subcoat and an enteric coat) was determined in a USP Type 2 apparatus, using USP apparatus type 2 at 50 rpm in media containing 0.1 N HCl for 2 hours, followed by pH 6.8 buffer for 22 hours.

| Time (hr) | % Release |
|---|---|
| 0.0 | 0.0 |
| 0.5 | 0.0 |
| 1.0 | 0.0 |
| 2.0 | 0.0 |
| 4.0 | 5.6 |
| 6.0 | 15.1 |
| 8.0 | 26.2 |
| 10.0 | 36.0 |
| 12.0 | 47.6 |
| 14.0 | 60.7 |
| 16.0 | 81.5 |
| 18.0 | 90.6 |
| 20.0 | 98.5 |

Oxybutynin chloride extended release tablets in Example 2 (with subcoat and enteric coat) also have zero-order release characteristics with complete release of oxybutynin chloride in 24 hours.

The subcoating enhances the resistance of the tablet to long term thermal aging without altering the zero-order release characteristics of the tablets. The Opadry Clear coating is effective as a stabilizer when applied as a 10% solution to provide a weight gain of 2 wt % based on the total weight of the uncoated tablet prior to enteric coating.

1. A compressed tablet of a pharmaceutical compound which comprises uncoated pellets containing a pharmaceutical compound, said pellets being dispersed in a matrix which comprises said pellets and a swellable polymer which is compressed into a tablet that is coated with an enteric polymer.

2. A compressed tablet of a pharmaceutical compound as defined in claim 1 wherein the uncoated pellets contain a pharmaceutical excipient.

3. A compressed tablet of a pharmaceutical compound as defined in claim 2 wherein the pharmaceutical excipient is selected from the group consisting of microcrystalline cellulose, dicalcium phosphate, calcium sulfate, talc, silicon dioxide and calcium carbonate.

4. A compressed tablet of a pharmaceutical compound as defined in claim 1 wherein the swellable polymer is selected from the group consisting of carbomer, hydroxy propyl cellulose, hydroxypropyl methylcellulose and polyvinylpyrrolidone.

5. A compressed tablet of a pharmaceutical compound as defined in claim 2 wherein the swellable polymer is carbomer.

6. A compressed tablet of a pharmaceutical compound as defined in claim 4 wherein the swellable polymer is carbomer.

7. A compressed tablet of a pharmaceutical compound as defined in claim 1 where the enteric polymer is selected from the group consisting of shellac, methacrylic acid copolymers, cellulose acetate phthalate, hydroxypropyl methylcellulose phthalate, hydroxypropyl methylcellulose acetate succinate, cellulose acetate trimellitate and polyvinyl acetate phthalate.

8. A compressed tablet of a pharmaceutical compound as defined in claim 7 where the enteric polymer is a methacrylic acid copolymer.

9. A compressed tablet of a pharmaceutical compound as defined in claim 1 which includes a subcoat under the enteric coating.

10. A compressed tablet of a pharmaceutical compound which comprises uncoated pellets containing said pharmaceutical compound and microcrystalline cellulose and dicalcium phosphate; said pellets being dispersed in a matrix which comprises said uncoated pellets, a carbomer and microcrystalline cellulose which is compressed into a tablet that is coated with a methacrylic acid containing enteric polymer.

11. A compressed tablet of a pharmaceutical compound as defined in claim 10 which includes a subcoat under the enteric coating.

12. A method of making a tablet of a pharmaceutical compound which comprises;
(a) preparing uncoated pellets containing a pharmaceutical compound;
(b) dispersing said uncoated pellets in a matrix which comprises a swellable polymer;
(c) compressing said matrix into a tablet; and
(d) coating said tablet with an enteric polymer.

13. A method of making a tablet of a pharmaceutical compound as defined in claim 12 wherein the uncoated pellets contain a pharmaceutical excipient.

14. A method of making a tablet of a pharmaceutical compound as defined in claim 13 wherein the pharmaceutical excipient is selected from the group consisting of microcrystalline cellulose, dicalcium phosphate, calcium sulfate, talc, silicon dioxide and calcium carbonate.

15. A method of making a tablet of a pharmaceutical compound as defined in claim 12 wherein the swellable polymer is selected from the group consisting of carbomer, hydroxy propyl cellulose, hydroxypropyl methylcellulose and polyvinylpyrrolidone.

16. A method of making a tablet of pharmaceutical compound as defined in claim 13 wherein the swellable polymer is carbomer.

17. A method of making a tablet of a pharmaceutical compound as defined in claim 15 wherein the swellable polymer is carbomer.

18. A method of making a tablet of a pharmaceutical compound as defined in claim 12 where the enteric polymer is selected from the group consisting of shellac, methacrylic acid copolymers, cellulose acetate phthalate, hydroxypropyl methylcellulose phthalate, hydroxypropyl methylcellulose acetate succinate, cellulose acetate trimellitate and polyvinyl acetate phthalate.

19. A method of making a tablet of a pharmaceutical compound as defined in claim 18 where the enteric polymer is a methacrylic acid copolymer.

20. A method of making a tablet of a pharmaceutical compound which comprises;
(a) preparing uncoated pellets containing a pharmaceutical compound, microcrystalline cellulose and dicalcium phosphate;
(b) dispersing said uncoated pellets in a matrix which comprises a carbomer and microcrystalline cellulose;
(c) compressing said matrix into a tablet; and
(d) coating said tablet with an enteric polymer which comprises a methacrylic acid copolymer.

21. A method of making a pharmaceutical tablet as defined in claim 20 which includes the additional step of applying a subcoat under the enteric coating.

22. A compressed tablet of oxybutynin chloride which comprises uncoated pellets containing oxybutynin chloride, said pellets being dispersed in a matrix which comprises said pellets and a swellable polymer which is compressed into a tablet that is coated with an enteric polymer.

23. A compressed tablet of oxybutynin chloride as defined in claim 22 wherein the uncoated pellets contain a pharmaceutical excipient.

24. A compressed tablet of oxybutynin chloride as defined in claim 23 wherein the pharmaceutical excipient is selected from the group consisting of microcrystalline cellulose, dicalcium phosphate, calcium sulfate, talc, silicon dioxide and calcium carbonate.

25. A compressed tablet of oxybutynin chloride as defined in claim 22 wherein the swellable polymer is selected from the group consisting of carbomer, hydroxy propyl cellulose, hydroxypropyl methylcellulose and polyvinylpyrrolidone.

26. A compressed tablet of oxybutynin chloride as defined in claim 23 wherein the swellable polymer is carbomer.

27. A compressed tablet of oxybutynin chloride as defined in claim 25 wherein the swellable polymer is carbomer.

28. A compressed tablet of oxybutynin chloride as defined in claim 22 where the enteric polymer is selected from the group consisting of shellac, methacrylic acid copolymers, cellulose acetate phthalate, hydroxypropyl methylcellulose phthalate, hydroxypropyl methylcellulose acetate succinate, cellulose acetate trimellitate and polyvinyl acetate phthalate.

30. A compressed tablet of oxybutynin chloride as defined in claim 28 where the enteric polymer is a methacrylic acid copolymer.

31. A compressed tablet of oxybutynin chloride which comprises uncoated pellets containing oxybutynin chloride and microcrystalline cellulose and dicalcium phosphate; said pellets being dispersed in a matrix which comprises said uncoated pellets, a carbomer and microcrystalline cellulose polymer which is compressed into a tablet that is coated with a methacrylic acid containing enteric polymer.

32. A compressed tablet of a pharmaceutical compound as defined in claim 31 which includes a subcoat under the enteric coating.

33. A method of making a tablet of oxybutynin chloride which comprises;
(a) preparing uncoated pellets containing oxybutynin chloride;
(b) dispersing said uncoated pellets in a matrix which comprises a swellable polymer;
(c) compressing said matrix into a tablet; and
(d) coating said tablet with an enteric polymer.

34. A method of making a pharmaceutical tablet as defined in claim 33 which includes the additional step of applying a subcoat under the enteric coating.

35. A method of making a tablet of oxybutynin chloride as defined in claim 33 wherein the uncoated pellets contain a pharmaceutical excipient.

36. A method of making a tablet of oxybutynin chloride as defined in claim 33 wherein the pharmaceutical excipient is selected from the group consisting of microcrystalline cellulose, dicalcium phosphate, calcium sulfate, talc, silicon dioxide and calcium carbonate.

37. A method of making a tablet of oxybutynin chloride as defined in claim 33 wherein the swellable polymer is selected from the group consisting of carbomer, hydroxy propyl cellulose, hydroxypropyl methylcellulose and polyvinylpyrrolidone.

38. A method of making a tablet of oxybutynin chloride as defined in claim 35 wherein the swellable polymer is carbomer.

39. A method of making a tablet of oxybutynin chloride as defined in claim 37 wherein the swellable polymer is carbomer.

40. A method of making a tablet of oxybutynin chloride as defined in claim 33 where the enteric polymer is selected from the group consisting of shellac, methacrylic acid copolymers, cellulose acetate phthalate, hydroxypropyl methylcellulose phthalate, hydroxypropyl methylcellulose acetate succinate, cellulose acetate trimellitate and polyvinyl acetate phthalate.

41. A method of making a tablet of oxybutynin chloride as defined in claim 40 where the enteric polymer is a methacrylic acid copolymer.

42. A method of making a tablet of oxybutynin chloride which comprises;
(a) preparing uncoated pellets containing oxybutynin chloride, microcrystalline cellulose and dicalcium phosphate;
(b) dispersing said uncoated pellets in a matrix which comprises a carbomer and microcrystalline cellulose;
(c) compressing said matrix into a tablet; and
(d) coating said tablet with an enteric polymer which comprises a methacrylic acid copolymer.

43. A method of making a pharmaceutical tablet as defined in claim 42 which includes the additional step of applying a subcoat under the enteric coating.
